(19) United States
(12) Patent Application Publication
(10) Pub. No.: US 2015/0140644 A1
(43) Pub. Date: May 21, 2015

(54) TREATMENT OF LEAF TISSUE FOR THE RECOVERY OF INTERSTITIAL RECOMBINANT PROTEINS BY THE APPLICATION OF CELL WALL DEGRADING ENZYMES

(71) Applicant: The Regents of the University of Califomia, Oakland, CA (US)

(72) Inventors: Karen A. MCDONALD, Davis, CA (US); Nathaniel J. KINGSBURY, Davis, CA (US)

(21) Appl. No.: 14/547,022

(22) Filed: Nov. 18, 2014

Related U.S. Application Data
(60) Provisional application No. 61/905,812, filed on Nov. 18, 2013.

(51) Int. Cl. C12P 21/00; C12N 15/82

(52) U.S. Cl. CPC C12P 21/00; C12N 15/8257
USPC 435/272

(57) ABSTRACT

The present disclosure relates to methods of increasing the purity of an apoplast-targeted recombinant protein recovered from a plant tissue, and increasing the yield of recovery of an apoplast-targeted recombinant protein from a plant tissue.

### CROSS-REFERENCE TO RELATED APPLICATION

This application claims the benefit of U.S. Provisional Application No. 61/905,812, filed Nov. 18, 2013, which is hereby incorporated by reference in its entirety.

### STATEMENT REGARDING FEDERALLY SPONSORED RESEARCH OR DEVELOPMENT

This invention was made with government support under Grant No. 1067432, awarded by the National Science Foundation. The government has certain rights in this invention.

### FIELD

The present disclosure relates to methods of increasing the yield of recovery and increasing the purity of an apoplast-targeted recombinant protein recovered from a plant tissue.

### BACKGROUND

In planta production of apoplast-targeted cellulose degrading enzymes may be a valuable approach for efficient production of concentrated enzyme preparations that can be used for saccharification of cellulose in pretreated feedstocks. The use of agroinfiltration to produce heterologous proteins of interest (POI) in leaf tissue has been reviewed along with considerations affecting yield and downstream processing (Menkhaus T J et al., Biotechnology Progress, 20, 2004, 1001-1014). The benefits of obtaining a purified extract from plant tissue have been discussed previously (Hassan S et al., Plant Biotechnology Journal, 6, 2008, 733-748; Wilken L R et al., Biotechnology Advances, 30, 2012, 419-433), and a method for recovering apoplast wash fluid from leaf tissue has been described (Klement Z, Phytopathology, 55, 1965, 1033; Rathmell W G et al., Plant Physiology, 53, 1974, 317-318). However, traditional recovery methods, such as tissue homogenization, suffer from disadvantages including that high fiber content fouls chromatography columns, equipment is and operating costs are expensive, there is an increased likelihood of contaminant release, and air-liquid interfaces may damage protein (Hassan S et al., 2008, Plant Biotechnology Journal, 6, 733-748). Previous reports have used apoplast wash fluid (AWF) recovery to remove secreted recombinant POI from leaf tissue and quantified its recovery as a percent yield of the total protein expressed (Turpen T H et al., U.S.P. Office (Ed.), Large Scale Biology Corporation, USA, 2006, pp. 28; Lombardi R et al., Transgenic Research, 19, 2010, 1083-1097). However, protein degradation in leaf tissue, especially in the leaf apoplast, remains a major industrial impediment (Hehle V K et al., BMC Biotechnology, 2011; Doran P M, Trends in Biotechnology, 24, 2006, 426-432). Thus, there exists a need for improved methods of increasing the production and recovery of a protein of interest from the apoplast of plant tissues.

### BRIEF SUMMARY

This disclosure offers an improvement on existing methods for recovering secreted (apoplast-targeted) recombinant proteins from agroinfiltrated leaf tissue. It has been discovered that recovery of a protein of interest can be improved by using cell wall degrading enzymes (CWDE). Without wishing to be bound by theory, it is believed that use of cell wall degrading enzymes allows the pore size of the plant cell wall to increase, allowing large proteins or protein bodies to flow through more readily and enter AWF whereas without treatment they would have been inaccessible to the procedure. The hydrodynamic radius of proteins of a given molecular weight can be approximated by empirical formulas (H. P. Erickson, Biological Procedures Online, 2009). The porosity of plant cell walls may also be measured by a variety of methods and has been found for some species (Titel, C., et al., Planta, 1997). With this information, it can be calculated by how much the diffusion of particles with is limited through a porous material (Phillips, R. J., Biophysical Journal, 2000). As the size of the proteins is on the same order of magnitude as the size of the cell wall pores, an increase in pore size could have an exponential effect on the protein's effective diffusivity.

The protein of interest tested for this study was butyrylcholinesterase, a decoy protein protecting against sarin gas attacks, but this invention should also apply to any secreted protein of interest, particularly those whose transport through the extracellular matrix of leaf tissue is limited by pore size. AWF recovery was used for the removal of proteins as it was optimal for achieving high yield, purity, and concentration of extracts relative to whole tissue homogenization, but it is imagined that treatment by CWDE would facilitate recovery by other non-destructive methods as well, such as passive diffusion or repeated vacuum infiltrations. Non-destructive methods also may keep leaves alive and productive after recovery of secreted POI, boosting overall expression levels, more effective removal of POI by CWDE treatment would further enhance the effectiveness of protein recovery from apoplasts.

In one aspect, the present disclosure relates to a method of producing butyrylcholinesterase (BuChE) in a leaf tissue, the method including: providing leaf tissue from Nicotiana tabacum transiently transformed to express BuChE under the control of the TRBO vector, incubating the leaf tissue such that the BuChE is expressed and located to an apoplast of a cell of the leaf tissue, contacting the leaf tissue with a cell-wall degrading enzyme, contacting the leaf tissue with a rinse fluid using vacuum infiltration-centrifugation to release the BuChE from the apoplast into the rinse fluid to create an apoplast wash fluid, where content of the BuChE in the apoplast wash fluid from leaf tissue treated with cell wall-degrading enzyme and contacted with rinse fluid is at least six-fold higher than the content of the BuChE in the apoplast wash fluid from a comparable leaf tissue not treated with cell wall-degrading enzyme and purity of the the BuChE in the apoplast wash fluid from leaf tissue treated with cell wall-degrading enzyme and contacted with rinse fluid is at least two-fold higher than the purity of the BuChE in the apoplast wash fluid from a comparable leaf tissue not treated with cell wall-degrading enzyme.

In another aspect, the present disclosure relates to a method of recovering a recombinant protein in a plant tissue, the method including: a first step of providing plant tissue transformed to express recombinant protein; a second step of contacting plant tissue with cell-wall degrading enzyme; a third step of extracting apoplast-targeted recombinant protein from plant tissue; a fourth step of incubating the plant tissue for an incubation period; and a fifth step of extracting apoplast-targeted recombinant protein from plant tissue, wherein the fifth step extracts recombinant protein synthesized after the third step.

In another aspect, the present disclosure relates to a method including: contacting the plant tissue with a cell wall-degrading enzyme and contacting the plant tissue with a rinse fluid on a plurality of occasions over the course of a production interval to release the recombinant protein from the apoplast into the rinse fluid to create an apoplast wash fluid, where content of the recombinant protein in the apoplast wash fluid from plant tissue contacted with rinse fluid on a plurality of occasions is higher than the content of the recombinant protein in the apoplast wash fluid from comparable plant tissue contacted with rinse fluid only at the end of the production interval.

In some embodiments, the plant tissue is transiently transformed with a nucleic acid encoding a recombinant protein in operable combination with a promoter.

In some embodiments, the method includes the step of incubating the plant tissue under suitable conditions such that the recombinant protein is expressed and located to an apoplast of a plant cell of the plant tissue.

In some embodiments, the incubation interval is 1 hour, 12 hours, 1 day, or 2 days.

In some embodiments, the cell wall-degrading enzyme is selected from the group including cellulases, hemicellulases, pectinases, endoglucanases, exoglucanases, and other cell-wall degrading or cell-wall modifying proteins

In some embodiments, the cell wall-degrading enzyme is pectinase.

In some embodiments, combinations of a variety of classes of cell wall-degrading enzymes are used.

In some embodiments, the recombinant protein is any large protein or proteins that tend to conglomerate into oligomers or larger protein bodies.

In some embodiments, the protein is butyrylcholinesterase.

In some embodiments, the plant tissue is a leaf tissue.

In some embodiments, the plant tissue is from Nicotiana benthamiana or Nicotiana tabacum.

In some embodiments, the leaf tissue is from N. tabacum.

In some embodiments, the plant tissue is stably transformed.

In some embodiments, the plant tissue is transiently transformed using Agrobacterium.

In some embodiments, the plant tissue is transiently transformed using viral vectors.

In some embodiments, the promoter is a CaMV 35S promoter.

In some embodiments, the contacting step includes vacuum-infiltrating the plant tissue to produce a vacuum-infiltrated plant tissue submerged in the rinse fluid.

In some embodiments, the method further includes centrifuging the vacuum-infiltrated plant tissue to facilitate separation of the apoplast wash fluid from the plant tissue.

In some embodiments, force of the centrifuging step is not more than 30 kPa.

In some embodiments, centrifugation occurs for not more than 20 minutes.

In some embodiments, the rinse fluid includes a protein-stabilization agent.

In some embodiments, each occasion of the plurality of occasions occurs at a regular periodic interval over the course of the production interval.

In some embodiments, the regular periodic interval is about once every 24 hours over the course of the production interval.

In some embodiments, the production interval is about 6 days.

In some embodiments, at least a portion of the plant tissue remains viable after each occasion of the plurality of occasions.

In some embodiments, viable plant tissue remains capable of expressing the recombinant protein.

In some embodiments, content of the recombinant protein in the apoplast wash fluid from plant tissue contacted with rinse fluid on a plurality of occasions is at least two-fold higher than the content of the recombinant protein in the apoplast wash fluid from comparable plant tissue contacted with rinse fluid only at the end of the production interval.

In some embodiments, purity of the recombinant protein in the apoplast wash fluid from plant tissue contacted with rinse fluid on a plurality of occasions is at least 125-fold higher than the purity of the recombinant protein in the apoplast wash fluid from comparable plant tissue contacted with rinse fluid only at the end of the production interval.

In some embodiments, the method further includes recovering the recombinant protein from the apoplast wash fluid.

### DESCRIPTION OF THE FIGURES

FIG. 1 The yield and purity of butyrylcholinesterase (BuChE) recovered after one round of vacuum infiltration-centrifugation on tissue treated with three different concentrations of pectinase. For each sample set, n=3. The asterisk indicates a significantly different set with a confidence interval of ≧99%.

FIG. 2 The yield of reducing sugar detected in the pooled AWF samples recovered by three rounds of VI-C from tissue treated with 0.15%, 0.015% or 0% v/v pectinase buffer. The asterisk indicates a significantly different set with a confidence interval of ≧99%.

FIG. 3 The purity of the model protein compared to that of an intracellular marker observed in the AWF after one round of recovery at 250 g for sample sets infiltrated with different amounts of pectinase enzyme. Sample size is n=3 for each set.

FIG. 4 Shows yields of BuChE accumulated after each round of recovery (RF and AWF combined). Differences between 0.15% and either 0.015% or 0% pectinase treated leaves were significant.

FIG. 5 The accumulated percent yield and pooled purification-fold improvement of peroxidase recovered in RF and AWF from tissue excised from triplicate 12 week old (wo) N. tabacum leaves over five rounds of vacuum infiltration-centrifugation. a) Accumulated percent yield after each round of recovery. b) Purity vs. yield chart over five rounds of recovery, depicted for each leaf sample.

FIG. 6A The accumulated recovery (combined AWF and RF) of truncated E1 (catalytic domain) and the full-length E1 endoglucanase after each round of infiltration-centrifugation. Differences in yield between catalytic domain and full-length protein were significant at each round. FIG. 6B Accumulated recovery of the truncated E1cd collected after each of three rounds of infiltration-centrifugation. Purity is graphed as an improvement over the homogenate extract on one axis and its purity per milligram of total soluble protein on the other axis. Yield is graphed both as percent yield and also as the units of enzyme recovered per kg fresh weight of leaf tissue.

FIG. 7 The purity and yield of BuChE recovered after one round of vacuum infiltration-centrifugation on tissue treated with three different concentrations of pectinase. For each sample set, n=3. The asterisk indicates a significantly different set with a confidence interval of ≧99%.

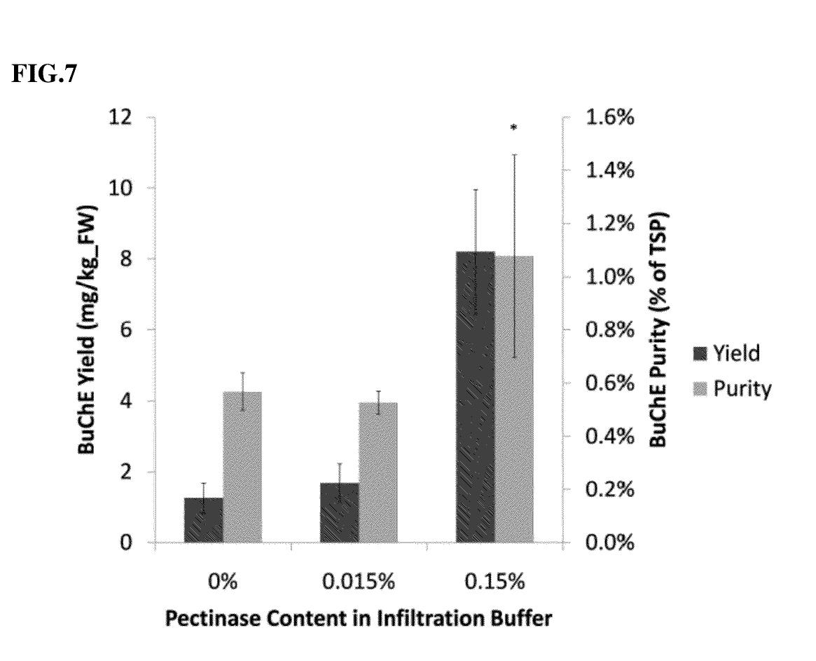

FIG. 8. In leaf tissue with AWF recovered daily, the percent yields of E1cd activity, malate dehydrogenase (MDH) activity, and total soluble protein (TSP) accumulated in AWF after daily recovery, reported as a percent of each sample's overall expression level (n=3).

### DETAILED DESCRIPTION

The present disclosure relates to methods of increasing the yield and/or recovery of an apoplast-targeted recombinant protein from a plant tissue and increasing the purity of an apoplast-targeted recombinant protein recovered from a plant tissue.

In particular, the present disclosure is based, at least in part, on Applicant's discovery that providing cell wall-degrading enzymes, prior to or concurrently with, multiple rounds of periodic rinsing of plant tissues to release a protein from the apoplast, increases protein purity in the rinsed plant tissue, as well as results in increased protein recovery from the apoplast. The methods of the present disclosure employ non-destructive recovery methods to remove proteins of interest from leaf tissue while preserving the leaf tissue for its further production. These methods described herein may find use in improving protein yields, improving protein purity, and potentially improving leaf health.

The present disclosure provides a method wherein plant tissue expressing apoplast-targeted recombinant protein may be treated with a cell wall-degrading enzyme prior to protein extraction and recovery of the recombinant protein may be increased over recovery from plants that have not been treated with a cell wall-degrading enzyme prior to protein extraction. Recovery using the disclosed method may be increased at least two-, three-, four-, five-, six-, seven-, eight-, nine-, or at least ten-fold over recovery from plants that have not been treated with a cell wall-degrading enzyme prior to protein extraction. Purity of proteins recovered from cell wall-degrading enzyme-treated plant tissue may be increased at least two-fold over proteins recovered from plants that have not been treated with a cell wall-degrading enzyme prior to protein extraction. Purity of proteins recovered using the disclosed method may be increased at least two-, three-, four-, or at least five-fold over proteins recovered from plants that have not been treated with a cell wall-degrading enzyme prior to protein extraction.

The terms “decrease,” “reduce” and “reduction” as used in reference to biological function (e.g., enzymatic activity, production of compound, expression of a protein, etc.) refer to a measurable lessening in the function by preferably at least 10%, more preferably at least 50%, still more preferably at least 75%, and most preferably at least 90%. Depending upon the function, the reduction may be from 10% to 100%. The term “substantial reduction” and the like refers to a reduction of at least 50%, 75%, 90%, 95% or 100%.

The terms “increase,” “elevate” and “elevation” as used in reference to biological function (e.g., enzymatic activity, production of compound, expression of a protein, etc.) refer to a measurable augmentation in the function by preferably at least 10%, more preferably at least 50%, still more preferably at least 75%, and most preferably at least 90%. Depending upon the function, the elevation may be from 10% to 100%; or at least 10-fold, 100-fold, or 1000-fold up to 100-fold, 1000-fold or 10,000-fold or more. The term “substantial elevation” and the like refers to an elevation of at least 50%, 75%, 90%, 95% or 100%.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that expressed the protein. As such an isolated protein is free of extraneous or unwanted compounds (e.g., nucleic acids, native bacterial proteins, etc.).

As used herein, the singular form “a”, “an”, and “the” includes plural references unless indicated otherwise. For example, “a rinse” includes one or more rinses.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting” and/or “consisting essentially of” aspects and embodiments.

The term “non-destructive techniques” should be understood as meaning any method that removes just the apoplast from the leaf tissue while leaving the rest of the tissue in place. Typically, this is performed by flooding the interstitium with buffer solution, tailored to optimal stability of the POI, allowing the POI to diffuse into the buffer, and then either allowing the POI to diffuse out of the leaf or by forcing the infiltrated buffer out to dry the tissue. The preferred method of infiltrating buffer into leaf tissue is by a vacuum chamber, as this can process many samples of leaf tissue in parallel. The duration, strength, and number of vacuum applications would vary based on the species and quality of tissue used.

The description presented herein is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Transformation of Plant Tissue with Recombinant Proteins

The methods of the present disclosure involve transient transformation of a plant tissue to express a recombinant protein. Methods of transient transformation are well-known in the art and are described herein. Exemplary methods include the Agrobacterium tumefaciens transformation system. Suitable vectors for use in the transformation system are also well-known in the art. Transient transformation systems with plant tissues typically involve transforming a plant cell or tissue to express a recombinant nucleic acid operably linked to a promoter to drive expression of the nucleic acid. Methods of constructing recombinant nucleic acids and promoters are well-known in the art and are described herein. In some embodiments, plant tissues are transiently transformed to express a recombinant nucleic acid encoding an E1 endoglucanase operably linked to the CaMV 35S promoter. In some embodiments, the plant tissue to be transiently transformed in leaf tissue. Suitable conditions to facilitate transient transformation of the plant tissue are well-known in the art such as, for example, incubating the agroinfiltrated plant tissue at 20° C. in a humid environment.

Various recombinant proteins may be used in the methods of the present disclosure. Suitable recombinant proteins include those that are secreted to, targeted to, expressed in, or otherwise present in the apoplast of a plant cell. In some embodiments, the recombinant protein is butyrylcholinesterase. Other exemplary proteins include, for example, those proteins that are useful in saccharification processes, such as cellulases, hemicellulases, pectinases, endoglucanases, exoglucanases, and other cell-wall degrading or cell-wall modifying proteins.

### Recovering Apoplast-Targeted Recombinant Protein

The methods of the present disclosure recover apoplast-targeted recombinant protein using a method having least one step of contacting plant tissue with a cell-wall degradation enzyme. Typically, the method involves a first step of providing plant tissue transformed to express recombinant protein; a second step of contacting plant tissue with cell-wall degrading enzyme; a third step of extracting apoplast-targeted recombinant protein from plant tissue; a fourth step of incubating the plant tissue for an incubation period; and a fifth step of extracting apoplast-targeted recombinant protein from plant tissue, wherein the fifth step extracts recombinant protein synthesized after the third step. In certain embodiments, the method includes a sixth step of contacting plant tissue with cell-wall degradation enzyme. The enzyme may either be the same or different as that in the second step. Without wishing to be bound by theory, Applicants believe that use of CWDE at a suitable concentration enhances protein recovery through a two-fold effect: a) increasing pore size of the cell wall, which allows larger proteins to enter the apoplast wash fluid more readily and b) doing so without impairing protein synthesis, so protein production continues while proteins are being extracted.

Treatment with Cell Wall-Degrading Enzymes

The methods of the present disclosure involve treating plant tissue with a cell wall-degrading enzyme (CWDE) to improve the purity and yield of apoplast-localized recombinant proteins from plant tissue. While any CWDE that is effective breaking down fibers may be effective for this purpose, the preferred class is pectinase. Other classes of CWDE include cellulases, hemicellulases, pectinases, endoglucanases, exoglucanases, and other cell-wall degrading or cell-wall modifying proteins. It is desirable that the fiber targeted for degradation has an important role in cross-linking the extracellular matrix and is generally not resistant to enzymatic digestion in its native form within the leaf tissue. Cocktails of a variety of classes of CWDE may also be effective.

Without wishing to be bound be theory, Applicants believe that CWDE should be applied at concentrations which increase cell wall pore size to permit larger proteins to enter the apoplast wash fluid and/or which are sufficiently gentle to permit continued synthesis of recombinant protein. For pectinase, concentrations of CWDE may be around 0.15% volume/volume solution and may range from about 0.10% to about 0.2% or from about 0.05% to about 0.25%. The concentration will need to be adjusted for the particular CWDE used, and may be 0.01%, 0.015%, 0.1%, 0.15%, 0.2%, 0.3%, 0.4%, 0.5%, 0.6%, 0.7%, 0.8%, 0.9% or 1.0%.

Plant tissue is incubated with CWDE solution for an incubation period. Typically this incubation period is between 1 hour and 2 days, for example, 3 hrs, 6 hrs, 12 hrs, 1 day, or 2 days. The incubation may be for even longer, e.g., 3 days, 4 days, or 5 days. Subsequent to this incubation period, protein extraction techniques may be employed as known in the art. This CWDE incubation may occur only once prior to multiple rounds of protein extraction, may occur immediately prior to each round of protein extraction, or may occur concurrently with the protein extraction rounds.

### Extracting Apoplast-Targeted Recombinant Protein

Standard methods for extracting apoplast-targeted recombinant protein are well known in the art. Non-destructive techniques remove just the apoplast from the leaf tissue while leaving the rest of the tissue in place. Typically, this is performed by flooding the interstitium with buffer solution, tailored to optimal stability of the POI, allowing the POI to diffuse into the buffer, and then either allowing the POI to diffuse out of the leaf or by forcing the infiltrated buffer out to dry the tissue. The preferred method of infiltrating buffer into leaf tissue is by a vacuum chamber, as this can process many samples of leaf tissue in parallel. The duration, strength, and number of vacuum applications would vary based on the species and quality of tissue used.

### Rinsing Procedures

In certain embodiments, the methods of extracting apoplast-targeted recombinant protein involve periodic rinsing of a transiently transformed plant tissue to release proteins from the apoplast so that the recombinant proteins can be recovered. Rinsing may refer to a process or series of processes that facilitate the release of a protein from the apoplast of a plant cell in a plant tissue. The methods of the present disclosure make use of periodic rinses to increase the total content of protein that can be recovered.

Following transformation of the plant tissue and once protein expression and secretion begins, the apoplast is rinsed to recover its components. In some preferred embodiments, rinsing is performed using vacuum infiltration-centrifugation (VI-C). By the VI-C method, the leaves are submerged in a rinse fluid that should be optimized for protein stabilization. To facilitate the infiltration, the rinse fluid typically would contain low levels of non-ionic surfactant, such as Silwet L-77. Leaves may be infiltrated individually or as a set within a large vacuum chamber in any orientation, although it is preferred that leaves be completely submerged with their abaxial (bottom) side facing up to facilitate the removal of air bubbles from their stomata. Various types of vacuum chamber or pump may be appropriate, but the vacuum pressure that is achieved should be able to go below 50 kPa, and an absolute pressure of at most 30 kPa at most is preferred. Vacuum pressure may be applied and released several times with the leaf or leaves still submerged, or the duration of the vacuum pressure application can be optimized, because it is preferable to achieve the greatest infiltrated volume per gram of leaf tissue. If vacuum pressure is applied multiple times per round of infiltration or if the leaves are submerged for a total duration longer than five minutes, it is recommended that the rinse fluid is collected as it will contain secreted recombinant protein with high purity. Furthermore, other methods of rinse fluid infiltration, such as pressure infiltration with a syringe, would be sufficient to produce the desired result while preventing leakage of the recombinant protein into the rinse fluid.

When using the vacuum infiltration-centrifugation method, the second step of the VI-C method is centrifugation of the whole leaves. A variety of centrifuges and apparatuses may be used for collection of the resulting fluid, called apoplast wash fluid (AWF), as described previously (Turpen T H et al., U.S.P. Office (Ed.), Large Scale Biology Corporation, USA, 2006, pp. 28), but they all should satisfy the general principle of allowing the centrifugal force to pull and separate the AWF from the leaves. The apparatus used may be a perforated 50 mL Falcon tube containing 7-12 pinholes approximately 3 mm in diameter at its conical bottom. Pinhole size, orientation, and other parameters should not create pressure points on leaf and cause unnecessary damage to the tissue. Other designs may include hanging the leaf from the top of the tube using a hook or a clamp. The centrifugal force employed should be sufficient to recover apoplast wash fluid while not causing damage to the leaf, which can be in the form of crushing, tearing, or creasing. These damage marks, while largely acceptable when simply recovering AWF for a one-time extraction, can create necrotic regions in the tissue. To cause the least amount damage, leaves may be rolled into the form of a cylinder with its adaxial side face out. Then they may be folded lengthwise in half and loaded into the tube with the stem of the leaf resting on the tube's bottom. The stem itself may be cut as short as possible to the edge of the leafy tissue. The highest centrifugal force that could be employed in, for example, a Beckman GS-6KR centrifuge while consistently ensuring leaves were not damaged was 400*g for Nicotiana tabacum and 125*g for Nicotiana benthamiana. The duration of centrifugation may be as long as 20 minutes, after which it is unlikely that further AWF could be collected from the leaves at that force.

After the centrifugation, the leaf may look dry (containing light regions), but often infiltrated fluid remains in the leaf (dark regions). It is preferred in such instances to perform another round of VI-C to recover more AWF rather than increase the centrifugal force or risk damaging the leaf tissue. Indeed, as many rounds of VI-C per day as practical, such as 5 rounds of periodic rinsing on a single day, may be performed since the time required for each cycle is relatively short. Plant tissue that is periodically rinsed may remain viable and capable of continuing to produce recombinant protein. In some embodiments, the periodic rinsing occurs once every 24 hours. The frequency of periodic rinsing may be altered to be longer or shorter depending on the health of the sample leaves. When to perform the initial round of VI-C may also be optimized. For example, there may be improvements in the purity of the initial AWF and RF recovered if there was a round of VI-C prior to the agroinfiltration. In between intervals of periodic rinsing, and generally, leaves should remain incubated at 20° C. in a humid environment to prevent wilting and transpiration, which can negatively affect yield of AWF volume. The VI-C method may allow for 400 mL of rinse fluid per kg of fresh weight (but more typically, 300 mL/kg FW) to be moved into and out of, for example, a Nicotiana tabacum leaf tissue.

Periodic rinsing procedures using the methods of the present disclosure are performed over the course of a production interval. A production interval may refer to the period of time during which a transiently transformed plant tissue expresses a recombinant protein. In some embodiments, the production interval is 6 days and plant tissue is periodically rinsed every 24 hours (i.e. the plant tissue is rinsed a total of 6 times, once every 24 hours, over the course of 6 days). After each occasion of contacting the plant tissue with a rinse fluid using the rinsing procedures described herein, the apoplast wash fluid containing the recombinant protein isolated from the apoplast of a plant cell in the plant tissue may be recovered and various aspects of the recovered recombinant protein may be analyzed.

The periodic rinsing procedures of the present disclosure result in greater recombinant protein content in the apoplast wash fluid from plant tissue contacted with rinse fluid using periodic rinsing as compared to the content of the recombinant protein in the apoplast wash fluid from comparable plant tissue contacted with rinse fluid only at the end of the production interval. For example, when the plant tissue is rinsed a total of 6 times, once every 24 hours, over the course of 6 days (a 6 day production interval), the total recovered protein from the combination of all 6 recovered apoplast wash fluids is greater than the total recovered protein from plant tissue that was rinsed only on day six (the last day of the production interval). Periodic rinsing may also result in a higher protein content in the apoplast wash fluids from plant tissue subjected to periodic rinsing as compared to a comparable plant tissue where apoplast wash fluid was never recovered.

### EXAMPLES

To better facilitate an understanding of the embodiments of the disclosure, the following examples are presented. The following examples are merely illustrative and are not meant to limit any embodiments of the present disclosure in any way.

### Example 1

### Periodic Method for Recovering Recombinant Proteins from the Interstitial Fluid of Agroinfiltrated Nicotiana tabacum Leaves

The following Example describes a vacuum infiltration-centrifugation method developed for the recovery of transiently-produced proteins in the apoplast wash fluid (AWF) of tobacco leaves. The following example highlights the superior yield and purity of proteins obtained from plant tissue that was treated with cell wall-degrading enzymes prior to vacuum-infiltration centrifugation (VI-C) recovery of a recombinantly expressed protein.

### Results

In the results presented, agroinfiltration and incubation methods to produce butyrylcholinesterase (BCHE) is shown for Nicotiana benthamiana whole leaves, using the same methods as previously described (Kingsbury, N. J., et al., Biotechnology and Bioengineering, submitted). BCHE is a human blood glycoprotein that is found naturally in a tetrameric form with a size of about 340 kDa. In these studies BCHE was produced using transient vacuum agroinfiltration and targeted using a secretion signal peptide to the secretory system and ultimately to the apoplast. At the day of protein harvest, six days post-agroinfiltration, three sets of triplicate leaves were excised from the plant and treated for an hour by vacuum infiltrating buffer consisting of 0%, 0.015%, or 0.15% v/v pectinase solution. Leaves were then cut into strips and apoplast wash fluid (AWF) containing BCHE from tissue interstitium was recovered by three rounds of a vacuum infiltration-centrifugation method.

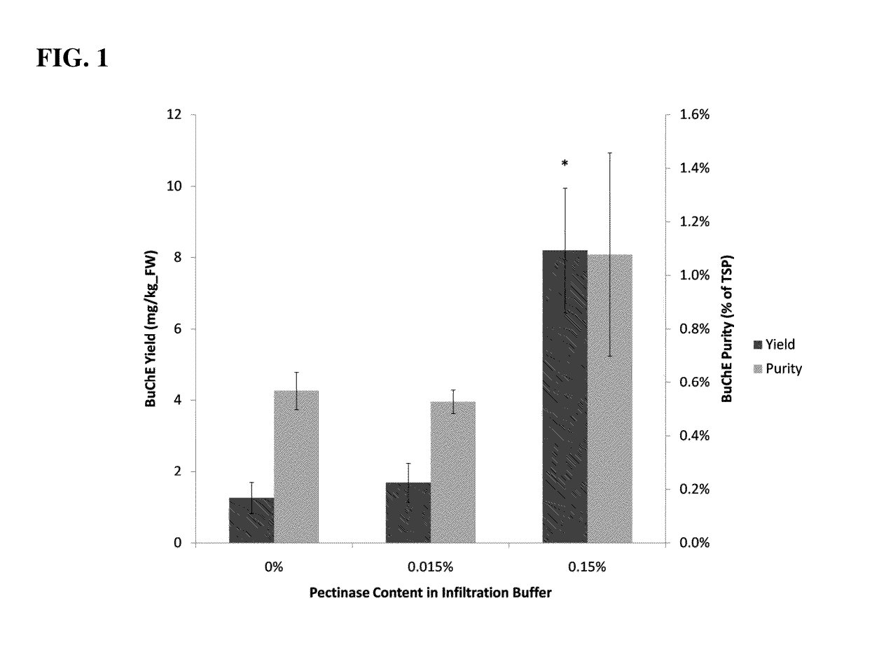

Treatment of butyrylcholinesterase (BuChE) expressing leaves with 0.15% pectinase solution prior to a round of vacuum infiltration-centrifugation improved yields recovered in AWF and RF by a factor of 6.5±2.8, up to 8.2±1.8 from 1.3±0.4 mg of BuChE per kg of fresh weight (Table 1). There was also an apparent increase in the purity of BuChE in the AWF and RF as a result of the pectinase treatment, as BuChE increased to 1.08%±0.38% of the TSP in the samples up from 0.57%±0.07%, a 1.9±0.7-fold improvement (FIG. 1). Meanwhile, application of a ten times relatively diluted pectinase solution of 0.015% had only a marginal if any effect on AWF or RF composition: increased BuChE yields by a factor of 1.4±0.7 and purity of 0.53%±0.04% (Table 2). Therefore, treatment with 0.15% pectinase solution tested best for recovering BuChE in AWF or RF at relatively higher yields and purity.

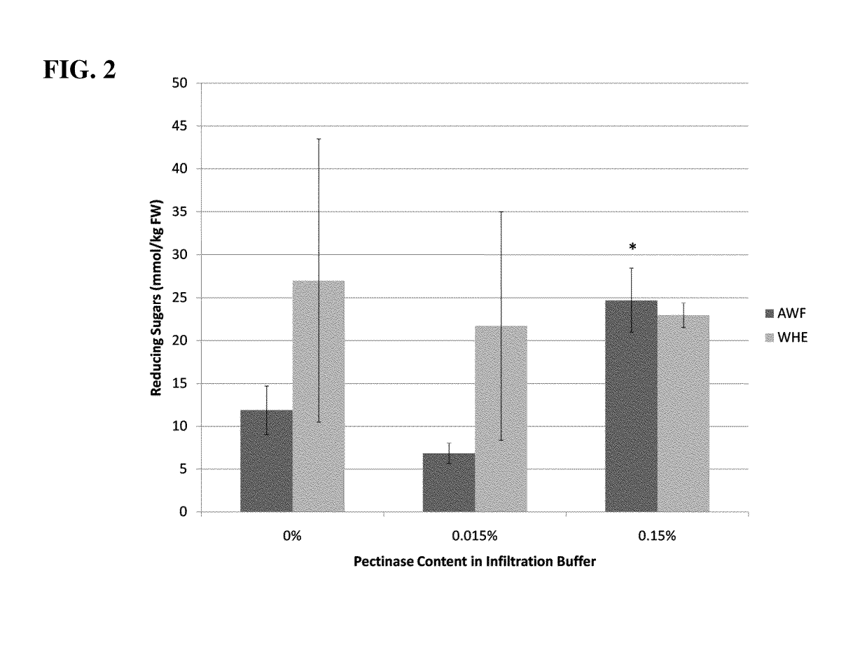

The yields of reducing sugar in the AWF of 0.15% pectinase treated tissue were significantly greater than those of 0.015% or 0% treated tissue, 25 mmol compared to 7 mmol or 12 mmol glucose equivalent per kg FW (FIG. 2). After three rounds of AWF recovery, the homogenate extract from pectinase treated tissue did not exhibit an increase in reducing sugars compared to that from non-treated tissue, so the sugars liberated during enzymatic treatment were thoroughly removed during the AWF recovery. This supported the claim that the invention was effective because pectinase broke down fiber in the extracellular matrix.

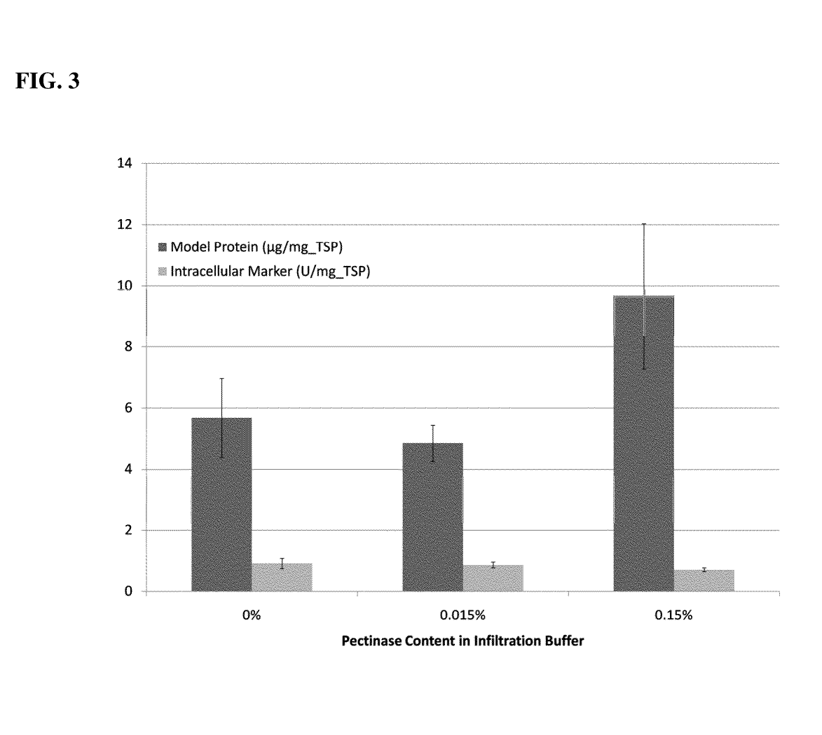

The invention was effective in purifying BuChE because it was selectively recovered by the vacuum infiltration-centrifugation method. When compared to the intracellular marker assayed, malate dehydrogenase (MDH), only BuChE showed an increase in purity upon the application of 0.15% v/v pectinase treatment (FIG. 3). In fact, MDH exhibited a slight decrease in purity as other large extracellular proteins were also liberated by pectinase treatment. These results were only valid at a centrifugal force 250 g because when the force was increased during the same cycle to 3200 g additional MDH was in fact released. However, the results showed that pectinase was effective in allowing diffusion of BuChE into AWF without cells breaking open at low centrifugal force. This offers further evidence that BuChE yields increased with pectinase treatment because it enhanced the permeability of the cell wall and not because of other factors such compromised tissue integrity.

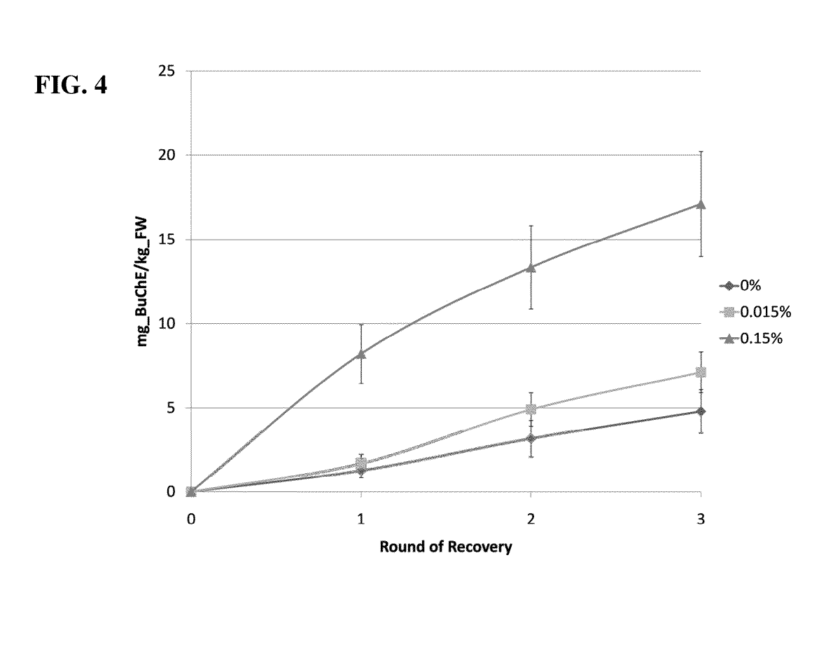

Pectinase treated leaf tissue continued to release BuChE into AWF and RF extracts upon application of second and third rounds of VI-C. Even though no further pectinase was introduced to the tissue after the initial treatment, this tissue continued to yield more BuChE per round of recovery than tissue treated with dilute or mock pectinase buffer, although returns diminished with each round (FIG. 4).

TABLE 1
The average yields of butyrylcholinesterase, total soluble protein, malate
dehydrogenase, and reducing sugars assayed from apoplast wash fluid, rinse fluid, and
homogenate extracts, reported for each of the three pectinase treatment sample sets.
0.15% Pectianse Treatment

| | | | | Reducing Sugars |
|---|---|---|---|---|
| Component | Butyrylcholinesterase | Total Soluble Protein | Malate Dehydrogenase | millimoles Glucose |
| Units | mg per kg FW | grams per kg FW | Enz. Units per kg FW | Eq. per kg |
| AWF1 | 7.05 ± 1.75 | 0.66 ± 0.10 | 0.86 ± 0.46 | 13.09 ± 1.75 |
| AWF2 | 3.31 ± 1.18 | 0.31 ± 0.14 | 0.49 ± 0.22 | 6.41 ± 3.09 |
| AWF3 | 2.72 ± 0.50 | 0.33 ± 0.14 | 0.76 ± 0.13 | 5.22 ± 1.17 |
| RF1 | 1.15 ± 0.00 | 0.12 ± 0.00 | 0.00 ± 0.00 | 0.00 ± 0.00 |
| RF2 | 1.83 ± 1.29 | 0.14 ± 0.07 | 0.00 ± 0.00 | 0.00 ± 0.00 |
| RF3 | 1.04 ± 0.52 | 0.13 ± 0.05 | 0.00 ± 0.00 | 0.00 ± 0.00 |
| WHE | 40.89 ± 7.65 | 6.52 ± 3.08 | 15.14 ± 2.85 | 22.95 ± 1.43 |
| TOTAL | 57.99 ± 8.08 | 8.22 ± 3.09 | 17.24 ± 2.90 | 47.66 ± 4.00 |

0.015% Pectinase Treatment

| Component | Butyrylcholinesterase | Total Soluble Protein | Malate Dehydrogenase | Reducing Sugars |
|---|---|---|---|---|
| Units | mg per kg FW | milligrams per kg FW | Enz. Units per kg FW | mmol per kg FW |
| AWF1 | 1.56 ± 0.54 | 0.29 ± 0.12 | 0.35 ± 0.16 | 2.37 ± 0.92 |
| AWF2 | 2.48 ± 0.79 | 0.25 ± 0.10 | 0.50 ± 0.16 | 2.68 ± 0.62 |
| AWF3 | 2.01 ± 0.42 | 0.24 ± 0.07 | 0.48 ± 0.21 | 1.77 ± 0.45 |
| RF1 | 0.13 ± 0.00 | 0.04 ± 0.00 | 0.00 ± 0.00 | 0.00 ± 0.00 |
| RF2 | 0.74 ± 0.24 | 0.09 ± 0.02 | 0.00 ± 0.00 | 0.00 ± 0.00 |
| RF3 | 0.20 ± 0.06 | 0.04 ± 0.01 | 0.00 ± 0.00 | 0.00 ± 0.00 |
| WHE | 40.53 ± 12.83 | 9.55 ± 3.49 | 15.11 ± 3.16 | 21.69 ± 13.31 |
| TOTAL | 47.64 ± 12.87 | 10.50 ± 3.49 | 16.44 ± 3.18 | 28.51 ± 13.36 |

0% Pectinase Treatment

| | | | | Reducing Sugars |
|---|---|---|---|---|
| Component | Butyrylcholinesterase | Total Soluble Protein | Malate Dehydrogenase | millimoles Glucose |
| Units | mg per kg FW | milligrams per kg FW | Enz. Units per kg FW | Eq. per kg |
| AWF1 | 1.14 ± 0.43 | 0.19 ± 0.02 | 0.56 ± 0.26 | 3.25 ± 1.80 |
| AWF2 | 1.56 ± 0.98 | 0.12 ± 0.04 | 0.26 ± 0.11 | 4.36 ± 1.57 |
| AWF3 | 1.48 ± 0.56 | 0.20 ± 0.05 | 0.30 ± 0.14 | 4.26 ± 1.53 |
| RF1 | 0.13 ± 0.00 | 0.03 ± 0.00 | 0.00 ± 0.00 | 0.00 ± 0.00 |
| RF2 | 0.35 ± 0.21 | 0.07 ± 0.01 | 0.00 ± 0.00 | 0.00 ± 0.00 |
| RF3 | 0.15 ± 0.06 | 0.04 ± 0.00 | 0.00 ± 0.00 | 0.00 ± 0.00 |
| WHE | 51.90 ± 25.82 | 10.73 ± 3.30 | 16.18 ± 2.47 | 27.00 ± 16.52 |
| TOTAL | 56.69 ± 25.85 | 11.39 ± 3.30 | 17.31 ± 2.49 | 38.87 ± 16.77 |

TABLE 2
Purity and concentration fold improvements and percent yields from N. benthamiana
tissue transiently expressing butyrylcholinesterase, reported for each of the
three pectinase treatment sample sets.

| Component | BuChE Activity | | | Total Soluble Protein | | | Malate Dehydrogenase | | | Reducing Sugars | | |
|---|---|---|---|---|---|---|---|---|---|---|---|---|
| Value | % Yield | Purity | Conc. | % Yield | Purity | Conc. | % Yield | Purity | Conc. | % Yield | Purity | Conc. |

0.15% Pectinase Treatment

| | | | | | | | | | | | | |
|---|---|---|---|---|---|---|---|---|---|---|---|---|
| AWF1 | 12.2% | 1.5 | 2.2 | 8.1% | — | 1.5 | 5.0% | 0.6 | 0.9 | 27.5% | 3.4 | 5.1 |
| AWF2 | 5.7% | 1.5 | 1.4 | 3.7% | — | 0.9 | 2.8% | 0.8 | 0.7 | 13.4% | 3.6 | 3.4 |
| AWF3 | 4.7% | 1.2 | 1.0 | 4.1% | — | 0.8 | 4.4% | 1.1 | 0.9 | 10.9% | 2.7 | 2.3 |
| RF1 | 2.0% | 1.4 | 0.0 | 1.5% | — | 0.0 | 0.0% | 0.0 | 0.0 | 0.0% | 0.0 | 0.0 |
| RF2 | 3.2% | 1.8 | 0.0 | 1.7% | — | 0.0 | 0.0% | 0.0 | 0.0 | 0.0% | 0.0 | 0.0 |
| RF3 | 1.8% | 1.1 | 0.0 | 1.6% | — | 0.0 | 0.0% | 0.0 | 0.0 | 0.0% | 0.0 | 0.0 |
| ΣAWF | 22.5% | 1.4 | 1.6 | 15.9% | — | 1.1 | 12.2% | 0.8 | 0.9 | 51.9% | 3.3 | 3.6 |
| ΣRF | 6.9% | 1.5 | 0.0 | 4.8% | — | 0.0 | 0.0% | 0.0 | 0.0 | 0.0% | 0.0 | 0.0 |
| WHE | 70.5% | 0.9 | 0.7 | 79.4% | — | 0.8 | 87.8% | 1.1 | 0.9 | 48.1% | 0.6 | 0.5 |

0.015% Pectinase Treatment

| | | | | | | | | | | | | |
|---|---|---|---|---|---|---|---|---|---|---|---|---|
| AWF1 | 3.3% | 0.8 | 0.7 | 2.7% | — | 0.6 | 2.1% | 0.8 | 0.5 | 8.3% | 3.1 | 1.8 |
| AWF2 | 5.2% | 1.4 | 1.1 | 2.4% | — | 0.5 | 3.1% | 1.3 | 0.7 | 9.4% | 4.0 | 2.1 |
| AWF3 | 4.2% | 1.2 | 0.9 | 2.3% | — | 0.5 | 2.9% | 1.3 | 0.6 | 6.2% | 2.8 | 1.3 |
| RF1 | 0.3% | 0.5 | 0.0 | 0.4% | — | 0.0 | 0.0% | 0.0 | 0.0 | 0.0% | 0.0 | 0.0 |
| RF2 | 1.6% | 1.1 | 0.0 | 0.9% | — | 0.0 | 0.0% | 0.0 | 0.0 | 0.0% | 0.0 | 0.0 |
| RF3 | 0.4% | 0.7 | 0.0 | 0.4% | — | 0.0 | 0.0% | 0.0 | 0.0 | 0.0% | 0.0 | 0.0 |
| ΣAWF | 12.7% | 1.7 | 0.9 | 7.3% | — | 0.5 | 8.1% | 1.1 | 0.6 | 23.9% | 3.3 | 1.7 |
| ΣRF | 2.2% | 1.3 | 0.0 | 1.7% | — | 0.0 | 0.0% | 0.0 | 0.0 | 0.0% | 0.0 | 0.0 |
| WHE | 85.1% | 0.9 | 0.9 | 91.0% | — | 0.9 | 91.9% | 1.0 | 0.9 | 76.1% | 0.8 | 0.8 |

0% Pectinase Treatment

| | | | | | | | | | | | | |
|---|---|---|---|---|---|---|---|---|---|---|---|---|
| AWF1 | 2.0% | 0.9 | 0.5 | 1.6% | — | 0.4 | 3.2% | 2.0 | 0.8 | 8.4% | 5.1 | 1.9 |
| AWF2 | 2.7% | 1.8 | 0.6 | 1.1% | — | 0.2 | 1.5% | 1.4 | 0.3 | 11.2% | 10.3 | 2.5 |
| AWF3 | 2.6% | 1.1 | 0.6 | 1.7% | — | 0.4 | 1.8% | 1.0 | 0.4 | 11.0% | 6.3 | 2.4 |
| RF1 | 0.2% | 0.5 | 0.0 | 0.3% | — | 0.0 | 0.0% | 0.0 | 0.0 | 0.0% | 0.0 | 0.0 |
| RF2 | 0.6% | 0.7 | 0.0 | 0.6% | — | 0.0 | 0.0% | 0.0 | 0.0 | 0.0% | 0.0 | 0.0 |
| RF3 | 0.3% | 0.5 | 0.0 | 0.4% | — | 0.0 | 0.0% | 0.0 | 0.0 | 0.0% | 0.0 | 0.0 |
| ΣAWF | 7.4% | 1.6 | 0.5 | 4.5% | — | 0.3 | 6.5% | 1.5 | 0.5 | 30.5% | 6.8 | 2.3 |
| ΣRF | 1.1% | 0.9 | 0.0 | 1.3% | — | 0.0 | 0.0% | 0.0 | 0.0 | 0.0% | 0.0 | 0.0 |
| WHE | 91.5% | 1.0 | 0.9 | 94.3% | — | 0.9 | 93.5% | 1.0 | 0.9 | 69.5% | 0.7 | 0.7 |

### Example 2

### Selective Recovery of Apoplast-Targeted Recombinant Proteins from Plant Leaf Tissue

Experiments were performed to demonstrate that VI-C can in fact be utilized to clear the leaf apoplast from the vast majority of secreted proteins. Peroxidase was studied as a native secretion marker protein, having been used to study AWF recovery in previous literature12, as a way to test the limits of the procedure. The experiment was then repeated with a heterologous protein, E1 endoglucanase catalytic domain (E1cd) from A. cellulolyticus, and its yields were compared to that of the larger (126 kDa vs. 51 kDa) full-length enzyme (E1). Next, cell wall degrading enzymes were employed to explore whether they could facilitate the recovery of a larger, more complex recombinant protein, recombinant human butyrylcholinesterase which forms a 340 kDa tetramer. Lastly, it was desired to see if AWF recovery could fulfill its promise as a non-destructive recovery technique: continued production of heterologous protein as product is being removed from the leaf. To this end, agroinfiltrated leaves had AWF recovery performed on them daily throughout the expression of E1cd and the overall expression levels in this tissue were compared to those using a single AWF recovery.

### Materials and Methods

Nicotiana benthamiana (GRIN Accession #: TW16)22 and Nicotiana tabacum var. Xanthii (from Bryce Falk Lab, UC Davis Plant pathology) were grown from seed in a greenhouse. Two-week old seedlings were transplanted three into 6″ pots.

The gene for full-length human butyrylcholinesterase (NCBI Accession #: AAA99296.1) was expressed using a Tobacco Mosaic Virus RNA-based overexpression (TRBO) vector (Lindbo J A. Plant Physiology. December 2007; 145(4):1232-1240). The construct also included a 3× FLAG® peptide tag (Sigma-Aldrich, St. Louis, Mo.) for affinity purification and the RAmy3D secretion signal peptide from the α-amylase gene from Oryza sativa (NCBI Accession #: M59351.1). The constructs were synthesized and transformed into Agrobacterium tumefaciens EHA105 pCH32. Agroinfiltration of leaf tissue and subsequent incubation was performed as described previously (Arzola L, et al. International Journal of Molecular Sciences, August 2011: 12(8); Plesha M A, et al. Biotechology Progress, 2007: 23(6)).

The gene for full-length E1 endoglucanase from Acidothermus cellulolyticus (NCBI Accession #: P54583) was codon-optimized using GeneDesigner software (version 1.1.4.1, DNA 2.0, Burlingame, Calif.) and the codon usage table for N. benthamiana (Nakamura Y. et al. Nucleic Acids Research. 2000: 28(1)). The 41 amino acid native signal peptide was removed from the N-terminus and replaced with the RAmy3D signal peptide from the α-amylase gene from Oryza sativa (NCBI Accession #: M59351.1). To the C-terminus, a 6-His tag was added. The construct was placed under the control of the CaMV 35S promoter. The sequence and the gene in entirety were submitted to GenBank (Accession #: HQ541433). The method for construction for the truncated E1 was the same as for the full-length protein except with the cellulose binding domain and linker region between domains removed. The constructs for both the full-length peptide and the truncated protein were synthesized and then transformed into Agrobacterium tumefaciens EHA105 pCH32.

A vacuum infiltration-centrifugation method was performed to recover rinse fluid (RF) and apoplast wash fluid (AWF) from the leaf tissue. Whole leaves or strips excised from the intercostal spaces were processed for this purpose (Rathmell W G, et al. Plant Physiology, 1974: 53(2)). Vacuum infiltration was performed in a Nalgene® container where the leaf is submerged in buffer at 20-25° C. but otherwise optimized for stabilizing the protein of interest. All infiltration buffers were spiked with 0.02% v/v of the surfactant Silwet L-77 (Lehle Seeds, Round Rock, Tex.) to facilitate vacuum infiltration. The buffer for peroxidase and E1 studies was 50 mM sodium acetate (pH=5.5) and 100 mM NaCl. The buffer for recovering BuChE was 20 mM Tris, 200 mM NaCl, pH=8. The buffer used during the vacuum infiltration was usually collected and assayed since significant quantities of secreted proteins have been observed to diffuse out of a submerged leaves (Hassan S, et al. Plant Biotechnology Journal, 2008: 6(7)). After the tissue was infiltrated, it was inserted into 50 mL Falcon tubes, with each tube possessing 8-15 circular perforations about 3 mm in diameter each. During centrifugation, the AWF flows through these holes and into a collection cap at the bottom of the tube. A Beckman GS-6KR (Beckman Coulter, Inc., Brea, Calif.) centrifuge with swing-out wells was used for all experiments. The centrifuge was set to run for 10-20 minutes at 20° C. Depending on the type of tissue used, the centrifugal forces applied ranged from 250 g to 3200 g. After all rounds of VI-C were completed, the tissue was homogenized to test for residual proteins. Homogenization was performed either with liquid nitrogen or with a GrindoMix GM 200 (Retsch Technology, Haan, Germany). The buffer used for homogenization was always identical to the buffer used for AWF recovery so observed activities between homogenate extracts and AWF samples could be accurately compared.

The vacuum infiltration-centrifugation was typically applied to experimental sample tissue three or more times consecutively. In the case of the periodic AWF recovery experiment, the method was applied to whole leaves once per day starting from the second day after agroinfiltration and ended on the seventh day. As leaf health was paramount in this experiment, leaves were protected from folding or creasing during centrifugation by rolling them in sheets of aluminum foil prior to insertion into the Falcon tubes, providing additional structural support for the leaves. In the experiment where cell wall degrading enzymes were used, a pectinase cocktail from Aspergillus niger glycerol solution (Sigma Product#:P4716; 30 mg/mL; ≧5 U/mg; optimal pH=4.0) was spiked into 50 mM acetic acid, 150 mM NaCl, 0.02% Silwet L77, pH=5.0 and vacuum infiltrated into leaf tissue. The leaves would incubate for an hour while the infiltrated buffer transpired, after which three rounds of VI-C with Tris buffer was performed as normal. The collected AWF was assayed by the DNS method (Bailey M J, et al. Journal of Biotechnology, 1992, 23(3)) to identify reducing sugars in comparison with an untreated control and confirm that a saccharification reaction took place. Assays for E1 endoglucanase activity (Ziegelhoffer T, et al., Molecular Breeding, 2001, 8(2)), BuChE activity (Ellman G L, et al., Biochemical Pharmacology, 1961, 7(2)), peroxidase (Gregory R P F, Biochemical Journal, 1966, 101(3)), total soluble protein (Bradford M M, Analytical Biochemistry, 1976, 72(1-2)) and malate dehydrogenase (Delannoy M, et al., Proteomics, 2008, 8(11); Ting I P, Archives of Biochemistry and Biophysics, 1968, 125(1)) were performed as previously described through well-established protocol.

### Results and Discussion

Recovery of Native Peroxidase Using a Vacuum-Infiltration Technique Improves Selective Recovery

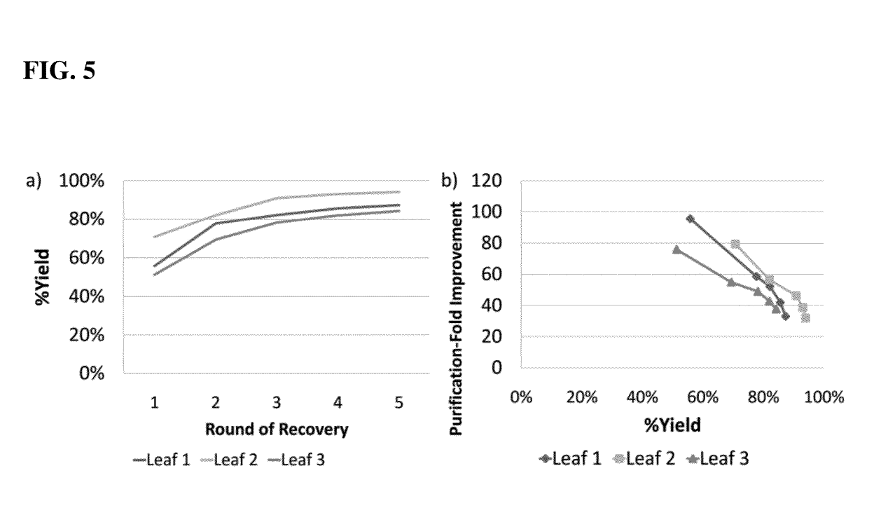

A vacuum infiltration-centrifugation (VI-C) technique to recover AWF and rinse fluid (RF) was performed on tissue excised from twelve-week-old N. tabacum leaves. Rinse fluid is the fluid that is recovered after the vacuum infiltration step (fluid that does not remain within leaf interstitial space). The activity of peroxidase was measured in the AWF compared to that of the residual homogenate extract (FIG. 5a). It was found that up to 94% of the peroxidase in these leaves could be recovered after five rounds of AWF recovery (19, 2.6, and 1.4 U/g FW measured in the AWF, RF, and residual homogenate extract respectively). The first round of recovery gave yields up to 71%, but each subsequent round was diluted by a factor of 4 on average. Total soluble protein (TSP) and malate dehydrogenase (MDH), a native intracellular plant protein used as an intracellular marker, were also measured, and it was found that for each round of recovery, about 2% of the tissue's TSP and less than 1% of the tissue's MDH was recovered, suggesting that peroxidase was in the extracellular space of the tissue and that this method was highly effective for its selective recovery of extracellular proteins. Indeed, for the first round of recovery, there was up to a 95-fold improvement in the peroxidase activity per mg TSP compared to homogenate extracts of “unwashed” tissue from the same leaf (400 vs. 4.2 U/mg TSP). This purification-fold improvement decreases as more extracts are pooled together over increasingly more rounds of VI-C, so increasing yields came at the expense of reduced purity of the extracts (FIG. 5b). AWF is also a much smaller volume than a homogenate extract, so peroxidase activity was as much as 16 times more concentrated that a homogenate (37 vs. 2.3 U/mL) extracted from the same leaf, performed by wet grinding at a standard buffer to biomass ratio of 10 mL per g FW.

Three rounds of vacuum infiltration-centrifugation were very effective recovering the recombinant E1 endoglucanase catalytic domain (E1cd) produced using transient agroinfiltration in N. benthamiana leaves (FIG. 4a). The activity of the apoplast wash fluid (AWF) was 1700±700 enzymatic units per kilogram of fresh weight in the leaf (U/kg FW; enzymatic unit is defined as 1 μmol of methylumbelliferone produced per minute). There was also activity observed in the infiltration buffer used during the vacuum step (RF), 500±100 U/kg FW. Meanwhile, there was only 600±400 U/kg FW remaining in the residual tissue. Therefore, the percent yield of E1cd recovered by AWF and RF in this experiment was 76.8%. Most of this yield was realized during the first round of recovery, with each subsequent round of recovery becoming diluted by a factor of 4±1. From this dilution factor, it was modeled that three rounds of recovery were sufficient, as additional rounds of recovery would only liberate an additional 2.5% of the overall expressed E1cd.

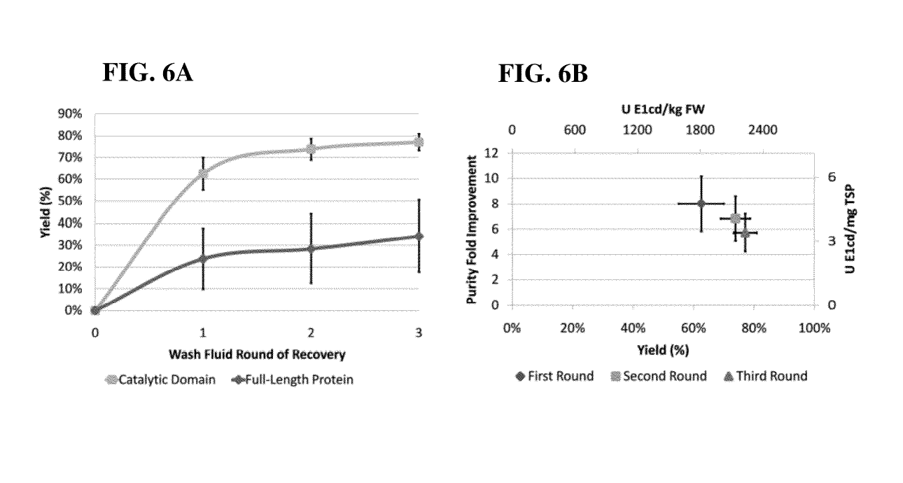

The yields of E1cd were higher than the yields for the full length E1 enzyme (E1). The overall expression level in the leaf tissue was 3.4 times that for leaves expressing E1. Furthermore, for the E1 protein, only 34.1%±16.5% of the overall activity expressed in the leaf was recovered in AWF or RF (FIG. 6A). There was high variability among the samples in this set and no trend observed was between yields from different rounds of recovery. It is therefore hypothesized that E1 experienced greater impedance from the extracellular matrix, either due to its cellulose binding domain or its larger hydrodynamic radius, which restricted its passage into the bulk fluid in the interstitium. This in turn would limit the full-length enzyme's diffusion into the RF or removal with the AWF.

The characteristic that most distinguishes AWF and RF extracts from homogenate extracts is its purity. From the first round of E1cd recovery, there was eight times more enzymatic activity per milligram of total soluble protein (TSP) than from the total overall expressed E1cd in the leaf tissue (FIG. 6B). This corresponded with very low (<1%) levels of the intracellular marker malate dehydrogenase, suggesting that purity improved because cells within the leaf were undisrupted throughout the vacuum infiltration and centrifugation steps. Second and third rounds of AWF recovery did generate additional yield of E1cd, but these extracts had less activity per mg TSP than the first recovered extracts.

Treatment with Cell Wall Degrading Enzymes Improves Yields by the Vacuum Infiltration-Centrifugation Method

N. benthamiana leaves agroinfiltrated to express and secrete butyrylcholinesterase (BuChE) to the apoplast were treated with cell wall degrading enzymes. Specifically, a pectinase cocktail was chosen to increase the size of the pores in the plant cell wall and liberate BuChE, which is known to assume several oligomeric forms including dimers and tetramers. AWF and RF was recovered from leaves incubated for an hour with vacuum infiltrated 0.15% v/v pectinase solution, and the yield was eight times greater than that from untreated tissue (8 mg/kg FW, up from 1 mg/kg FW) and six times greater than that from tissue treated with a ten-fold diluted pectinase solution (FIG. 1). This showed that pectinase did improve yields by non-destructive recovery techniques, and that these yields were sensitive to pectinase concentration. Furthermore, the increase in yield coincided with a two-fold increase in purity, increasing to 1.1% of the TSP recovered in the AWF compared to 0.6% for untreated tissue. Also, whereas the purity of the BuChE increased in 0.15% pectinase treated tissue increased from 6 to 10 μg per mg TSP, the activity of intracellular marker MDH remained constant at about 1 enzymatic unit per mg TSP. This suggested that the extra liberated protein recovered in AWF or RF by pectinase treatment was primarily secreted protein.

Apoplast Wash Fluid Recovery Performed Daily on Agroinfiltrated Leaves Improves Overall Productivity

Apoplast wash fluid (AWF) recovery was performed on tissue from N. benthamiana leaves agroinfiltrated to express E1cd. To facilitate comparison of tissue with AWF recovered daily versus tissue in which AWF was not recovered, vacuum infiltration was performed on only the right side (RS) of the leaf (abaxial or bottom side up) while the left side (LS) of the leaf remained dry. In this way, AWF was only recovered from the right sides of leaves. As an additional control another set of agroinfiltrated leaves were allowed to express E1cd unperturbed throughout the experiment. At eight days after agroinfiltration, the left and right sides of both sets of leaves were homogenized and their yields compared (Table 3). The yields from the tissue with AWF recovered daily were 106% higher than tissue from which no AWF was recovered. This was significantly greater than the difference in expression levels between the sides of the control leaves. In tissue with AWF recovered daily, as little as 8% of the total E1cd expressed remained in the tissue residually.

TABLE 3
E1cd expression levels (U/kg FW) in homogenate
extract and pooled AWF samples

| | Leaf side | Right side | | | RSvs. |
|---|---|---|---|---|---|
| | HE | HE | AWF | Total | LS |
| AWF Recovered Daily | | | | | |
| Leaf 1 | 4794 | 957 | 10404 | 11361 | 137% |
| Leaf 2 | 3958 | 836 | 7592 | 8427 | 113% |
| Leaf 3 | 1755 | 920 | 2053 | 2973 | 69% |
| Average | 3502 | 904 | 6683 | 7587 | 106% |
| AWF Not Recovered | | | | | |
| Leaf 1 | 6929 | 8081 | NC | 8081 | 17% |
| Leaf 2 | 3834 | 4757 | NC | 4757 | 24% |
| Leaf 3 | 2421 | 2720 | NC | 2720 | 12% |
| Average | 4395 | 5186 | — | 5186 | 18% |

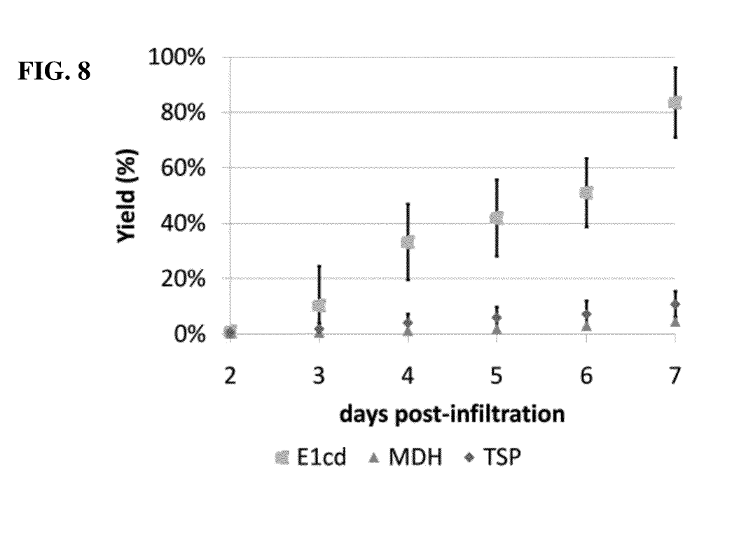

It was also shown in this experiment that yields of E1cd remained strong throughout the period of expression, two to seven days post-agroinfiltration (dpi). The most E1cd was recovered at 4 dpi, where it is expected transcription of the vector was at its peak, and also at 7 dpi, where changes in leaf morphology may have aided in the recovery of AWF (FIG. 8). This was also the day where the most TSP was recovered, but MDH only saw a marginal increase at 7 dpi, suggesting the extra liberated protein was primarily secreted. In all, 84%±13% of E1cd was recovered from leaves whereas only 11%±5% of TSP and 4%±1% of MDH were recovered by AWF from the leaf tissue during the course of the experiment. E1cd was also the only component tested of the three that exhibited an increase in overall expression level.

### CONCLUSIONS

The method of apoplast wash fluid recovery has been developed to recover native peroxidase and heterologous E1 endoglucanase catalytic domain, producing enriched extracts free from insoluble material and soluble intracellular molecules. Treatment of agroinfiltrated leaf tissue with cell wall degrading enzymes also showed the potential to recover larger proteins that may be restricted by structure the cell wall matrix. The native secretion pathway of plant cells is a powerful way to separate heterologous proteins from the cells that produce them, and apoplast wash fluid recovery harnesses that power and simplifies extraction and purification from leaf tissue. It also enabled higher overall productivity, perhaps by reducing feedback inhibition processes.

### REFERENCES

T. J. Menkhaus, Y. Bai, C. M. Zhang, Z. L. Nikolov, C. E. Glatz, Considerations for the recovery of recombinant proteins from plants, Biotechnology Progress, 20 (2004) 1001-1014.
S. Hassan, c. J. van Dolleweerd, F. loakeimidis, E. Keshavarz-Moore, J. K. C. Ma, Considerations for extraction of monoclonal antibodies targeted to different subcellular compartments in transgenic tobacco plants, Plant Biotechnology Journal, 6 (2008) 733-748.
L. R. Wilken, Z. L. Nikolov, Recovery and purification of plant-made recombinant proteins, Biotechnology Advances, 30 (2012) 419-433.
Z. Klement, METHOD OF OBTAINING FLUID FROM INTERCELLULAR SPACES OF FOLIAGE AND FLUIDS MERIT AS SUBSTRATE FOR PHYTOBACTERIAL PATHOGENS, Phytopathology, 55 (1965) 1033-&.
W. G. Rathmell, L. Sequeira, SOLUBLE PEROXIDASE IN FLUID FROM INTERCELLULAR SPACES OF TOBACCO-LEAVES, Plant Physiology, 53 (1974) 317-318.
Turpen T H, Garger S J, McCulloch M J, Cameron T I, Samonek-Potter M L, Holtz R B; Large Scale Biology Corporation, assignee. 2006 Apr. 25, 2006. Method for Recovering Proteins from the Interstitial Fluid of Plant Tissues. U.S. Pat. No. 7,034,128.
R. Lombardi, M. E. Villani, M. Di Carli, P. Brunetti, E. Benvenuto, M. Donini, Optimisation of the purification process of a tumour-targeting antibody produced in N. benthamiana using vacuum agroinfiltration, Transgenic Research, 19 (2010) 1083-1097.
V. K. Hehle, M. J. Paul, P. M. Drake, J. K. C. Ma, C. J. Van Dolleweerd, Antibody degradation in tobacco plants: a predominantly apoplastic process, in: BMC Biotechnology, 2011.
P. M. Doran, Foreign protein degradation and instability in plants and plant tissue cultures, Trends in Biotechnology, 24 (2006) 426-432.
D. D. Kuta, L. Tripathi, Agrobacterium-induced hypersensitive necrotic reaction in plant cells: a resistance response against Agrobacterium-mediated DNA transfer, African Journal of Biotechnology, 4 (2005) 752-757.
E. James, D. R. Mills, J. M. Lee, Increased production and recovery of secreted foreign proteins from plant cell cultures using an affinity chromatography bioreactor, Biochemical Engineering Journal, 12 (2002) 205-213.
T. Ziegelhoffer, J. A. Raasch, S. Austin-Phillips, Dramatic effects of truncation and subcellular targeting on the accumulation of recombinant microbial cellulase in tobacco, Molecular Breeding, 8 (2001) 147-158.
Arzola L, Chen J X, Rattanaporn K, Maclean J M, McDonald K A. Transient Co-Expression of Post-Transcriptional Gene Silencing Suppressors for Increased in Planta Expression of a Recombinant Anthrax Receptor Fusion Protein. International Journal of Molecular Sciences. August 2011; 12(8):4975-4990.
Conrad U, Fiedler U. 1998. Compartment-specific accumulation of recombinant immunoglobulins in plant cells: an essential tool for antibody production and immunomodulation of physiological functions and pathogen activity. Plant Molecular Biology 38(1-2):101-109.
Delannoy M, Alves G, Vertommen D, Ma J, Boutry M, Navarre C. 2008. Identification of peptidases in Nicotiana tabacum leaf intercellular fluid. Proteomics 8(11):2285-2298.
Geldner N. 2004. The plant endosomal system—its structure and role in signal transduction and plant development. Planta 219(4):547-560.
Gregory R P F. 1966. A rapid assay for peroxidase activity. Biochemical Journal 101(3):582-&.
Hegde R S, Bernstein H D. 2006. The surprising complexity of signal sequences. Trends in Biochemical Sciences 31(10):563-571.
Hellwig S, Drossard J, Twyman R M, Fischer R. 2004. Plant cell cultures for the production of recombinant proteins. Nature Biotechnology 22(11):1415-1422.
Lindbo J A. TRBO: A high-efficiency tobacco mosaic virus RNA-Based overexpression vector. Plant Physiology. December 2007; 145(4):1232-1240.
Lindenmuth B E, McDonald K A. 2011. Production and characterization of Acidothermus cellulolyticus endoglucanase in Pichia pastoris. Protein Expression and Purification 77(2):153-158.
Lohaus G, Pennewiss K, Sattelmacher B, Hussmann M, Muehling K H. 2001. Is the infiltration-centrifugation technique appropriate for the isolation of apoplastic fluid? A critical evaluation with different plant species. Physiologia Plantarum 111(4):457-465.
Nakamura Y, Gojobori T, Ikemura T. 2000. Codon usage tabulated from international DNA sequence databases: status for the year 2000. Nucleic Acids Research 28(1):292-292.
Nausch H, Mischofsky H, Koslowski R, Meyer U, Broer I, Huckauf J. 2012. Expression and Subcellular Targeting of Human Complement Factor C5a in Nicotiana species. Plos One 7(12).
Paciorek T, Sauer M, Balla J, Wisniewska J, Friml J. 2006. Immunocytochemical technique for protein localization in sections of plant tissues. Nature Protocols 1(1):104-107.
Plesha M A, Huang T-K, Dandekar A M, Falk B W, McDonald K A. High-level transient production of a heterologous protein in plants by optimizing induction of a chemically inducible viral amplicon expression system. Biotechnology Progress. November-December 2007; 23(6):1277-1285.
Rathmell W G, Sequeira L. 1974. Soluble peroxidase in fluid from intercellular spaces of tobacco leaves. Plant Physiology 53(2):317-318.
Sharp J M, Doran P M. 2001. Strategies for enhancing monoclonal antibody accumulation in plant cell and organ cultures. Biotechnology Progress 17(6):979-992.
Smith J M, Van Ness H C, Abbott M M. 2005. Vapor/Liquid Equilibrium: Introduction. Introduction to Chemical Engineering Thermodynamics. Singapore: McGraw Hill. p 338-370.
Surpin M, Raikhel N. 2004. Traffic jams affect plant development and signal transduction. Nature Reviews Molecular Cell Biology 5(2):100-109.
Terry M E, Bonner B A. 1980. An examination of centrifugation as a method of extracting an extracellular solution from peas, and its use for the stud of indoleacetic acid-induced growth. Plant Physiology 66(2):321-325.
Ting I P. 1968. Malic Dehydrogenases in Corn Root Tips. Archives of Biochemistry and Biophysics 126(1):1-&.
Trudel J, Potvin C, Asselin A. 1995. Secreted hen lysozyme in transgenic tobacco—recovery of bound enzyme and in-vitro growth inhibitions of plant-pathogens. Plant Science 106(1):55-62.
USDA A, National Genetic Resources Program. 2012. PI 555478 Nicotiana benthamiana Domin Solanaceae. 1962 ed.
Tzfira T, Citovsky V. Agrobacterium-mediated genetic transformation of plants: biology and biotechnology. Current Opinion in Biotechnology. April 2006; 17(2):147-154.
McDonald K A. In Planta Production of Cell Wall Degrading Enzymes. Handbook of Bioenergy Crop Plants 2011: 55-73.
Twyman R M, Stoger E, Schillberg S, Christou P, Fischer R. Molecular farming in plants: host systems and expression technology. Trends in Biotechnology. December 2003; 21(12):570-578.
Faye L, Boulaflous A, Benchabane M, Gomord W, Michaud D. Protein modifications in the plant secretory pathway: current status and practical implications in molecular pharming. Vaccine. March 2005; 23(15):1770-1778.
Paccalet T, Bardor M, Rihouey C, et al. Engineering of a sialic acid synthesis pathway in transgenic plants by expression of bacterial NeuSAc-synthesizing enzymes. Plant Biotechnology Journal. January 2007; 5(1):16-25.
Gomord W, Sourrouille C, Fitchette A C, et al. Production and glycosylation of plant-made pharmaceuticals: the antibodies as a challenge. Plant Biotechnology Journal. March 2004; 2(2):83-100.
Evangelista R L, Kusnadi A R, Howard J A, Nikolov Z L. Process and economic evaluation of the extraction and purification of recombinant beta-glucuronidase from transgenic corn. Biotechnology Progress. July-August 1998; 14(4):607-614.
Arntzen C, Mahoney R, Elliot A, et al. Plant-dervied Vaccines: Cost of Production. Tempe, Ariz.: The Biodesign Institute at Arizona State University; 2006.
Klement Z. Method of obtaining fluid from intercellular spaces of foliage and fluids merit as substrate for phytobacterial pathogens. Phytopathology. 1965; 55(9):1033-&.
Rathmell W G, Sequeira L. Soluble peroxidase in fluid from intercellular spaces of tobacco leaves. Plant Physiology. 1974; 53(2):317-318.
Surpin M, Raikhel N. Traffic jams affect plant development and signal transduction. Nature Reviews Molecular Cell Biology. February 2004; 5(2):100-109.
Bailey M J, Biely P, Poutanen K. Interlaboratory testing of methods for assay of xylanase activity. Journal of Biotechnology. May 1992; 23(3):257-270.
Ellman G L, Courtney K D, V. Andres J, Featherstone R M. A new and rapid colorimetric determination of acetylcholinesterase activity. Biochemical Pharmacology. 1961; 7(2):88-95.
H. P. Erickson, Size and Shape of Protein Molecules at the Nanometer Level Determined by Sedimentation, Gel Filtration, and Electron Microscopy, Biological Procedures Online, 11 (2009) 32-51.
C. Titel, H. Woehlecke, I. Afifi, R. Ehwald, Dynamics of limiting cell wall porosity in plant suspension cultures, Planta, 203 (1997) 320-326.
R. J. Phillips, A hydrodynamic model for hindered diffusion of proteins and micelles in hydrogels, Biophysical Journal, 79 (2000) 3350-3353.
N. J. Kingsbury, K. A. McDonald, Improved yields of secreted proteins from tobacco leaves using the vacuum infiltration-centrifugation method Biotechnology and Bioengineering In submission (2013) 18.
R. Fischer, Y. C. Liao, J. Drossard, Affinity-purification of a TMV-specific recombinant full-size antibody from a transgenic tobacco suspension culture, Journal of Immunological Methods, 226 (1999) 1-10.

1. A method for recovering apoplast-targeted recombinant protein from plant tissue, the method comprising:
a first step of providing plant tissue transformed to express an apoplast-targeted recombinant protein;
a second step of contacting the plant tissue with a cell wall-degrading enzyme;
a third step of extracting the apoplast-targeted recombinant protein from the plant tissue;
a fourth step of incubating the plant tissue for an incubation period; and
a fifth step of extracting the apoplast-targeted recombinant protein from the plant tissue,
wherein the fifth step extracts the apoplast-targeted recombinant protein synthesized after the third step.

2. The method of claim 1, wherein the third step of extracting the apoplast-targeted recombinant protein from the plant tissue comprises contacting the plant tissue with a rinse fluid to release the apoplast-targeted recombinant protein from the apoplast into the rinse fluid to create an apoplast wash fluid.

3. The method of claim 1, wherein the third step of extracting the apoplast-targeted recombinant protein from the plant tissue comprises contacting the plant tissue with a rinse fluid on a plurality of occasions over the course of a production interval to release the apoplast-targeted recombinant protein from the apoplast into the rinse fluid to create an apoplast wash fluid, where the content of the apoplast-targeted recombinant protein in the apoplast wash fluid from the plant tissue contacted with the rinse fluid on the plurality of occasions over the course of the production interval is higher than the content of the apoplast-targeted recombinant protein in the apoplast wash fluid from comparable plant tissue contacted with the rinse fluid only at the end of the production interval.

4. The method of claim 1, wherein the method further comprises a sixth step of contacting the plant tissue with the cell wall-degrading enzyme.

5. The method of claim 1, wherein the cell wall-degrading enzyme is pectinase.

6. The method of claim 5, wherein the pectinase is at a concentration of about 0.15% v/v.

7. The method of claim 5, wherein the pectinase is at a concentration ranging from about 0.10% to about 0.2% or from about 0.05% to about 0.25%.

8. The method of claim 1, wherein the cell wall-degrading enzyme is selected from the group consisting of cellulases, hemicellulases, pectinases, endoglucanases, and exoglucanases.

9. The method of claim 1, wherein the incubation period is selected from the group consisting of 12 hours, 1 day, and 2 days.

10. The method of claim 1, wherein the plant tissue is leaf tissue.

11. The method of claim 1, wherein the plant tissue is transiently transformed using Agrobacterium.

12. The method of claim 1, wherein the apoplast-targeted recombinant protein is at least about 51 kDa to about 340 kDa.

13. The method of claim 1, wherein the apoplast-targeted recombinant protein forms an oligomer.

14. The method of claim 1, wherein the second step of contacting the plant tissue with a cell wall-degrading enzyme takes place at the same time as the third step of extracting the apoplast-targeted recombinant protein from the plant tissue.

15. The method of claim 1, wherein the amount of the apoplast-targeted recombinant protein recovered from the plant tissue is at least six-fold higher than the amount of the apoplast-targeted recombinant protein recovered from the plant tissue not treated with the cell wall-degrading enzyme.

16. The method of claim 1, wherein the purity of the apoplast-targeted recombinant protein recovered from the plant tissue is at least two-fold higher than the amount of the apoplast-targeted recombinant protein recovered from the plant tissue not treated with the cell wall-degrading enzyme.
